(19) United States
(12) Patent Application Publication
(10) Pub. No.: US 2021/0125291 A1
(43) Pub. Date: Apr. 29, 2021

(54) SYSTEM AND METHOD FOR QUANTITATIVE NET PAY AND FLUID DETERMINATION FROM REAL-TIME GAS DATA

(71) Applicant: Chevron U.S.A. Inc., San Ramon, CA (US)

(72) Inventors: Mayank Malik, Houston, TX (US); Scott Alan Hanson, Houston, TX (US); Simon Robert John Clinch, Houston, TX (US)

(21) Appl. No.: 17/077,223

(22) Filed: Oct. 22, 2020

Related U.S. Application Data
(60) Provisional application No. 62/924,943, filed on Oct. 23, 2019.

(51) Int. Cl. G06Q 50/06; E21B 49/00; G01N 33/28; G01N 15/08

(52) U.S. Cl. CPC G06Q 50/06; E21B 49/005; G01N 15/088; G01N 33/2823; G01N 33/2841

(57) ABSTRACT

A method is described for quantitative pay summaries from mud logs, real-time fluid typing, and to minimize logging expense in high-angle or horizontal wells. The method may include receiving at least one mud log; selecting extraction system type, operating, and drilling parameters for the at least one mud log; determining hydrocarbon parameters such as methane (C1) extraction efficiency, trap response factor (TRF), and relative responses for ethane-pentane (C2-C5); correcting gas-in-mud volume to an earth surface to generate a corrected C1-C5; calculating a gas-to-oil ratio (GOR) from the corrected C1-C5; calculating a reservoir gas volume from the gas-in-mud volume to an earth surface and the GOR; determining pay cutoffs; and generating least one pay summary. The method may be executed by a computer system.

### CROSS-REFERENCE TO RELATED APPLICATIONS

This application claims priority benefit from U.S. Provisional Patent Application 62/924,943, filed Oct. 23, 2019.

### STATEMENT REGARDING FEDERALLY SPONSORED RESEARCH OR DEVELOPMENT

Not applicable.

### TECHNICAL FIELD

The disclosed embodiments relate generally to techniques for quantitative net pay determination and fluid determination from mud logs (i.e., mud gas data) representative of hydrocarbon wells drilled into subterranean hydrocarbon reservoirs.

### BACKGROUND

In hydrocarbon exploration, appraisal, and production, it is desirable to reduce process time and monetary cost as much as possible by using all available data including data from other wells in a field. When it comes to log data acquisition, there is often a compromise between what is desirable, what is feasible, and what is viable. Wireline logging has traditionally been able to provide the most reliable estimation of petrophysical properties. However, adverse wellbore conditions and challenging conveyance can increase the cost and preclude the use of wireline tools. Logging while drilling (LWD) can give the first glimpses of near-bit conditions but not all tools (such as multi-arm calipers, focused fluid sampling, rotary sidewall coring) are technically feasible in the drilling environment. By contrast, mud logging is in the sweet spot when it comes to data acquisition. Throughout the drilling process, mud logs provide real-time correlations with logs from adjacent wells and help track the bit's position in relation to target formations. Because the mud log is based on physical samples, it provides direct, independent identification of lithology, and hydrocarbon content. This information can be helpful when formation characteristics make wireline or LWD log interpretation complicated, ambiguous, or impractical. Thus, mud logging provides an independent, robust, and comprehensive understanding of reservoir geology and fluids.

There exists an opportunity for a method to use mud logs for the determination of quantitative net pay (i.e., estimation of how much of the rock formation traversed by a well contains hydrocarbons) and fluid determination (i.e., brine, gas, oil) in hydrocarbon reservoirs.

### SUMMARY

In accordance with some embodiments, a method of quantitative net pay determination and fluid determination from mud logs is disclosed. The method may include receiving at least one mud log from at least one well drilled through a subterranean rock formation containing hydrocarbons; selecting extraction system type, operating, and drilling parameters for the at least one mud log; determining hydrocarbon parameters including at least one of methane (C1) extraction efficiency, trap response factor (TRF), and relative responses for ethane-pentane (C2-C5); correcting gas-in-mud volume to an earth surface for the at least one mud log to generate a corrected C1-C5; calculating a gas-to-oil ratio (GOR) from the corrected C1-C5; calculating a reservoir gas volume from the gas-in-mud volume to an earth surface and the GOR; determining pay cutoffs based on the reservoir gas volume; and generating least one pay summary for the at least one well based on the pay cutoffs. The method may determine the hydrocarbon parameters using a calibration run. In another embodiment, the method may determine the hydrocarbon parameters using empirical values. The method may calculate the gas reservoir volume using pressure-volume-temperature data for field fluids. In another embodiment, the method may calculate the gas reservoir volume using regional fluid data. The method may estimate permeability from the reservoir gas volume. The method may generate three pay summaries for a low case, mid case, and high case. The method may also perform real-time fluid typing.

In another aspect of the present invention, to address the aforementioned problems, some embodiments provide a non-transitory computer readable storage medium storing one or more programs. The one or more programs comprise instructions, which when executed by a computer system with one or more processors and memory, cause the computer system to perform any of the methods provided herein.

In yet another aspect of the present invention, to address the aforementioned problems, some embodiments provide a computer system. The computer system includes one or more processors, memory, and one or more programs. The one or more programs are stored in memory and configured to be executed by the one or more processors. The one or more programs include an operating system and instructions that when executed by the one or more processors cause the computer system to perform any of the methods provided herein.

### BRIEF DESCRIPTION OF THE DRAWINGS

FIG. 1 illustrates a flowchart of a method of quantitative net pay determination and fluid determination from mud logs, in accordance with some embodiments;

FIG. 2 illustrates a step of a method of quantitative net pay determination and fluid determination from mud gas data, in accordance with some embodiments;

FIG. 3 illustrates of a step of a method of quantitative net pay determination and fluid determination from mud logs, in accordance with some embodiments;

FIG. 4 illustrates of a step of a method of quantitative net pay determination and fluid determination from mud logs, in accordance with some embodiments;

FIG. 5 shows that the gas trap recovery of the heavier components becomes more difficult and the relative recovery of these components with respect to methane (C1) decreases;

FIG. 6 illustrates of a step of a method of quantitative net pay determination and fluid determination from mud logs, in accordance with some embodiments;

FIG. 7 is an example dataset relating core-to-log calibrated permeability with bulk volume of hydrocarbons colored by deep resistivity;

FIG. 8 shows an example of different oils identified using a method of quantitative net pay determination and fluid determination from mud logs, in accordance with some embodiments;

FIG. 9 shows a ternary plot of C1-C2-C3 from mudlogs identifying different fluid types using a method of quantitative net pay determination and fluid determination from mud logs, in accordance with some embodiments;

FIG. 10 shows a result of a method of quantitative net pay determination and fluid determination from mud logs, in accordance with some embodiments; and

FIG. 11 is a block diagram illustrating a mud log pay determination system, in accordance with some embodiments.

Like reference numerals refer to corresponding parts throughout the drawings.

### DETAILED DESCRIPTION OF EMBODIMENTS

Reference will now be made in detail to various embodiments, examples of which are illustrated in the accompanying drawings. In the following detailed description, numerous specific details are set forth in order to provide a thorough understanding of the present disclosure and the embodiments described herein. However, embodiments described herein may be practiced without these specific details. In other instances, well-known methods, procedures, components, and mechanical apparatus have not been described in detail so as not to unnecessarily obscure aspects of the embodiments.

This present invention is used for generating quantitative pay summaries from mud logs (i.e., mud gas data), real-time fluid typing, and to minimize logging expense in high-angle or horizontal wells.

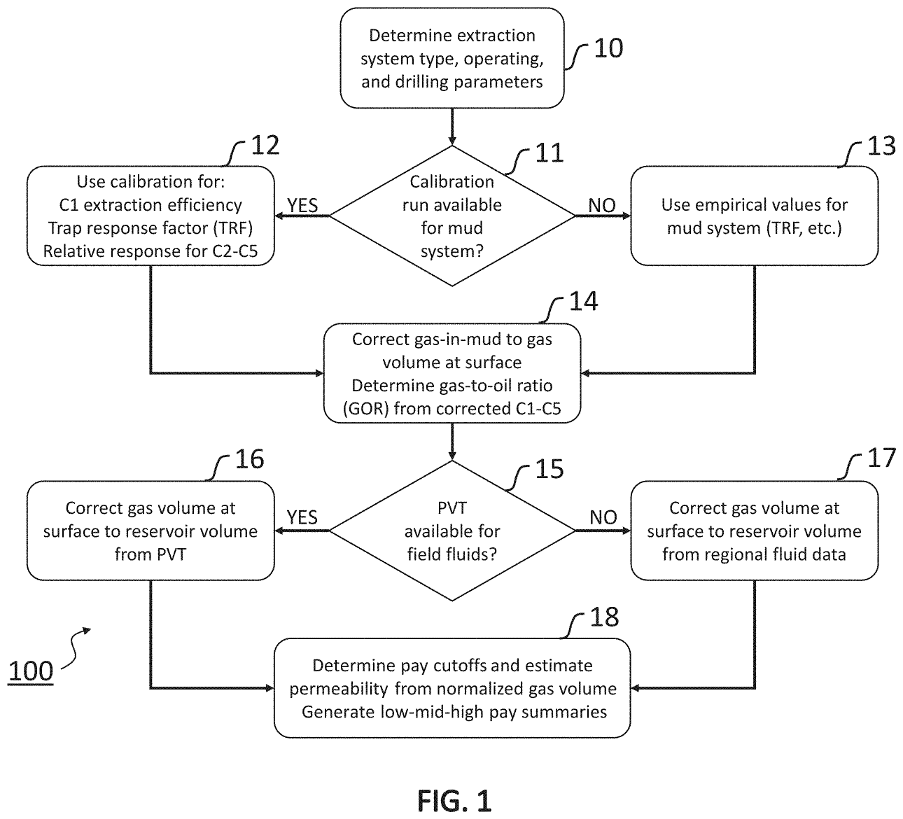

FIG. 1 illustrates a flowchart of a method 100 for quantitative net pay determination and fluid determination from mud logs. At operation 10, the type of extraction system, operating, and drilling parameters are determined This is important because different mud gas extraction systems used by various mudlogging providers have different mechanical operating parameters which can influence the gas data significantly. It is therefore necessary to normalized gas readings to provide a practical way to compare mud logs from different wells, even if the log data were produced by different mud logging companies.

At operation 11, the existence of a calibration run for the mud system is determined. If there is no calibration run 13, then empirical values are used for parameters of the mud system, for example the trap response factor (TRF). On the other hand, if a calibration run does exist 12, then those calibration results can be used to calculate the parameters including TRF, the extraction efficiency of C1 (methane), and the relative response for C2 (ethane)-C5 (pentane).

The traditional industry method of extracting hydrocarbons for analysis involves a gas extractor with an air-gas carrier system. Light gases methane (C1) through pentane (C5) are measured during drilling operation in parts per million (ppm). The gas values reported is gas-in-air (in %) or the gas concentration in air drawn from the head space of the gas extractor as

$$Gas - in - Air ( \% ) = \frac{{Σ}_{i = 1}^{n} {C}_{i}}{10000} \qquad ( 1 )$$

Quantitative petrophysical evaluation requires gas-in-mud or volumetric percent of gas in a volume of mud. The ratio of gas-in-air to gas-in-mud is the trap response factor (TRF). The relationship of gas-in-air to gas-in-mud can also be determined from the ratio of gas/air sample volume drawn from the gas trap relative to the volume of mud pumped through the gas trap and an estimate of extraction efficiency (EEC) for each gas component. Thus, the trap response factor for methane can be defined as:

$$T R {F}_{C 1} = \frac{Mud Flow Rate * EE {C}_{C 1}}{Suction Rate} \qquad ( 2 )$$

Knowing the mud flow through the gas trap, gas/air sample volume and extraction efficiency is critical in determining actual gas-in-mud fluid concentrations.

Efficiencies for methane through pentane extracted from oil-based mud systems may be determined based on extraction tests provided by mud logging companies. One such method for determining TRF is to circulate a volume of mud/gas through the gas extractor several times until no more gas can be extracted from the mud sample. (There is an assumption here that all the gas components have been removed from the mud sample). The gas values from each extraction cycle are then summed and compared to the initial value to determine the extraction efficiency. These efficiency tests are provided at the well site for the heated systems. Typically, efficiency tests are not performed on the non-heated volumetric mud extraction systems. The TRF for the QGM system was determined by extracting gas from a mud sample using a steam still or microwave system and comparing those values to values from the corresponding gas-in-air from the gas trap suction line. It should be noted that the steam still technique could be used on any gas measurement system but is not typically performed.

Correcting for recirculated gas is important because gas circulating out of the wellbore contains formation gas from intervals drilled but also gas that builds up and remains in the drilling fluid. For an oil-based mud, heavier gas components tend to build up and remain in the mud system more than the lighter gases. Thus, the amount of recirculated C3 to C5 can be a significant percentage of the total gas measured coming out of the well bore.

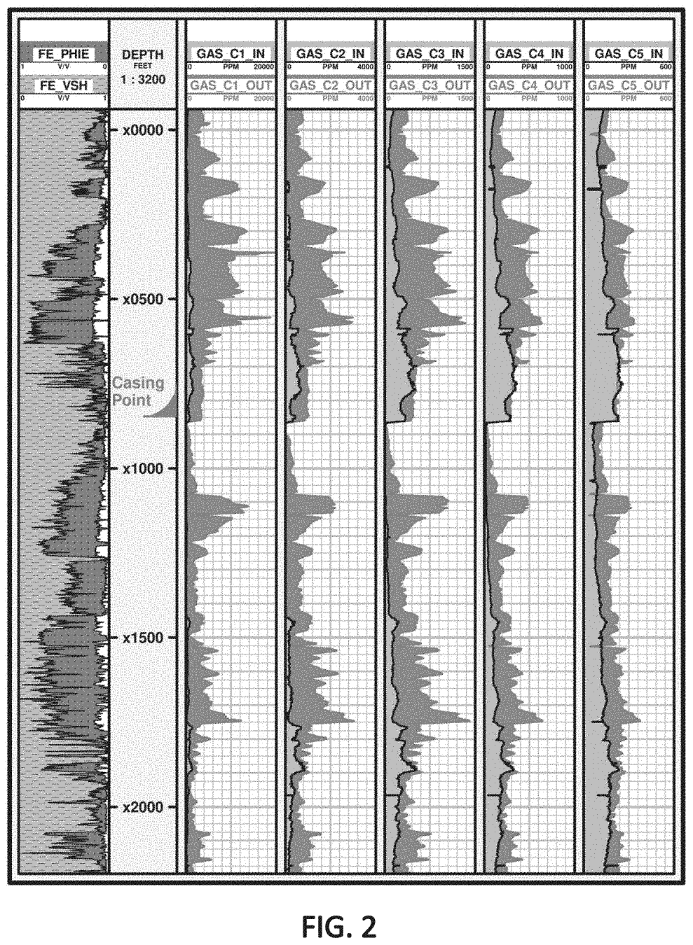

FIG. 2 shows gas values measured during drilling and includes C1-C5 measured in an oil-based mud system at flow-in and flow-out. The dark gray portion of the gas represents the gas coming from the formation. The light gray shaded portion is the recirculated gas. The plot shows that all gas components include some recirculated gas. Components heavier than propane contain up to 50% recirculated gas, even in high gas or potential show intervals. This data indicates that some means of correcting gas values for recirculation needs to be applied to characterize hydrocarbon using light gases measured during drilling. If gas values are not available from the suction pit or mud flow intake, then gas values should be corrected based on background values measured before the zone of interest.

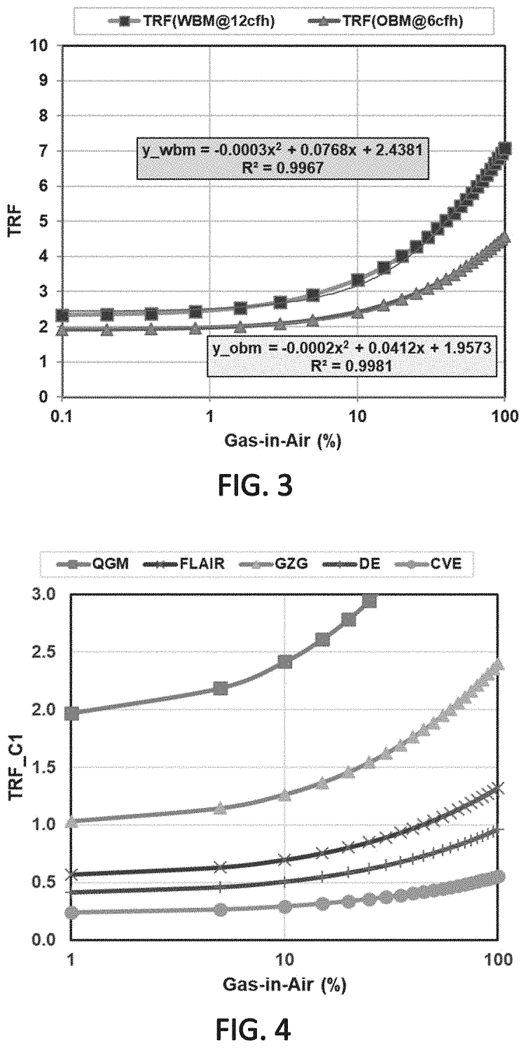

Trap response factors are important because each extractor is mechanically different and extracts gas differently from drilling fluids. FIG. 3 shows TRF based on flow loop and field tests for the QGM system in oil-based and water-based drilling fluid systems. Note the non-linear increase in TRF as gas-in-air values increase above about 6%. The gas trap system uses mechanical agitation to evolve gas from drilling fluids in a sealed extractor with internal baffles designed to limit immersion level effects.

The TRF is a ratio of total-gas-in-air (TG)/gas-in-mud (MG) and is derived from published equations for water-based mud (WBM) and oil-based mud (OBM). The TRF is applied to methane gas measured from the head space of the gas extractor for correction to actual gas values in the drilling fluid. This TRF is then adjusted to other stable gas extraction systems based on reported operating parameters. The TRF increases as gas-in-air values increase above about 6% because of increased efficiency of the extractor as the drilling fluid becomes aeriated with gas. The pump rate of the mud going into the gas trap and the suction rate of the gas going out of the gas trap affect the TRF. For this reason, the empirical equations shown in FIG. 3 are used to correct the non-linear effect.

Thus, the formula for the methane trap response factor for oil-base mud system is

$$T R {F}_{C 1} = \frac{{y}_{obm} * {TRF}_{sys}}{1.91} \qquad ( 3 )$$

and the formula for the methane trap response factor for water-base mud system is

$$T R {F}_{C 1} = \frac{{y}_{wbm} * T R {F}_{sys}}{2.32} \qquad ( 4 )$$

Where TRF_{sys} is the initial trap response factor of operating the mud system. FIG. 4 displays TRF curves plotted with total gas-in-air values for each of the systems. Corrected TRF_{C1} varies from 0.3 to 1.91 depending on the estimated extraction efficiency, the mud flow, and the suction rate.

Once TRF for methane is corrected, the next step is to calculate the relative response (RR) for other gas components as

$${RR}_{i} = \frac{T R {F}_{i}}{T R {F}_{C 1}} = \frac{E E {C}_{i}}{E E {c}_{C 1}} \qquad ( 5 )$$

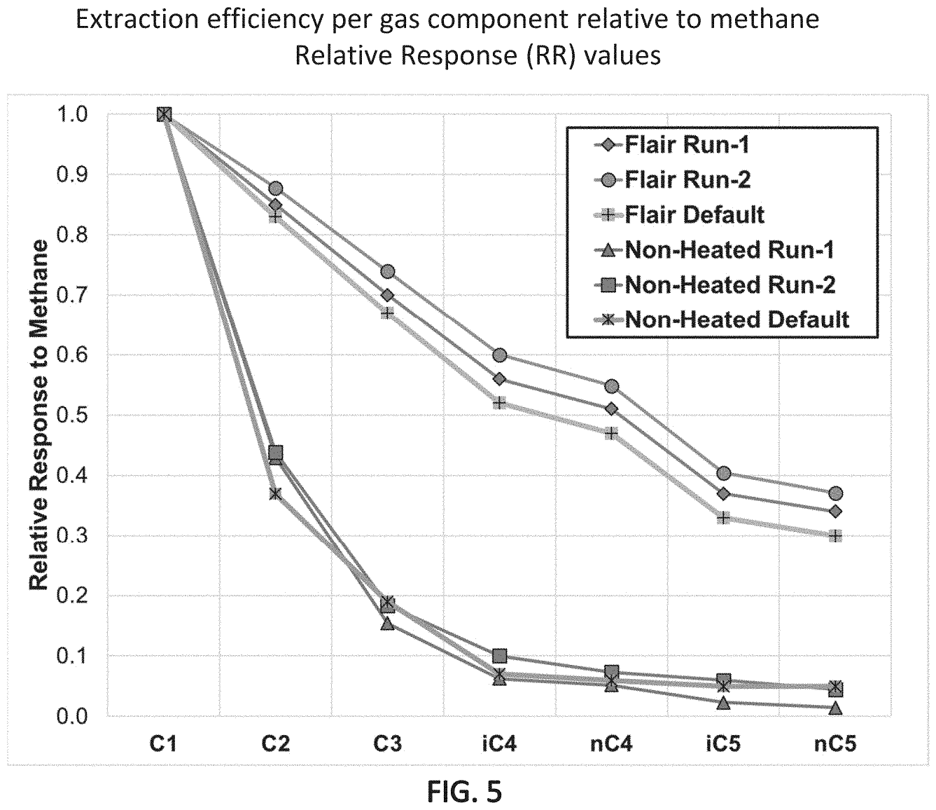

FIG. 5 displays relative response (RR) values for each gas component C1-C5. The RR values are the extraction efficiencies relative to methane for each of the gas components. The plot shows improved extraction efficiency for each gas component relative to methane from a heated system (FLAIR) when compared with non-heated system.

Referring again to FIG. 1, operation 14 corrects gas-in-mud to gas volume at surface and determine gas-to-oil ratio (GOR) from corrected C1-C5. Then it is determined if pressure-volume-temperature (PVT) data is available (operation 15) so that the method can either correct gas volume at surface to reservoir volume from PVT data if it exists (operation 16) or either correct gas volume at surface to reservoir volume from regional fluid data (operation 17).

At operation 16, the method calibrates mud log gas-oil ratio (GOR) with downhole fluid analysis and PVT samples, modifying trap response factors as needed. Trap response is the ratio of gas-in-air to gas-in-mud. Gas-in-air is the gas concentration in the sample line expressed as mole fraction (% gas/100). This is the trap gas value monitored in the logging unit. Gas-in-mud is the gas content of mud expressed as volume gas per volume of gas-free mud. This value is needed for mud log evaluation. For accurate evaluation, we need to know the trap response for each gas component measured. Trap calibration samples and PVT samples enable us to directly measure trap performance, primarily for tracking recovery of the heavier components.

After calculating gas-in-mud, the next step is to convert gas values to volume of gas measured at the surface per volume of rock drilled. This is done by environmentally correcting or normalizing the gas values for drill rate, drill bit size, and drilling fluid circulation rates. Normalized gas volume (NMGAS) is the ratio of the total gas measured at surface conditions (Vₛ) to the volume of cuttings drilled (V_{c}):

NMGAS=Vₛ/V_{c} (7)

Total gas measured at surface conditions in ft³ is

$${V}_{s} = ( \frac{\% Gas - in - Mud}{100} ) * {V}_{mud} * 0.133 \qquad ( 8 )$$

where % Gas is interval average gas-in-mud (after correcting with trap response factor) and V_{mud} is total volume of mud pumped in gallons. The multiplier of 0.133 is the unit conversion factor between gallons to ft³.

Volume of cuttings drilled in ft³ is

$${V}_{c} = {π ( \frac{BS}{2 * 12} )}^{2} * l \qquad ( 9 )$$

where BS is Bit Size or bit diameter in inches and l is length of interval drilled in feet.

Thus, normalized gas volume can be calculated by combining these three equations as

$$NMGAS = \frac{\% Gas - in - Mud * {V}_{m u d} * 0.245}{B {S}^{2} * l} \qquad ( 10 )$$

Bit Size controls the cross-sectional area of the wellbore and Rate of Penetration (ROP) controls the thickness of the interval drilled. These parameters, in turn, are affected by pump rate, weight on bit, rotary speed, fluid viscosity, and mud density. If the ROP in feet per hour and mud flow rates (FLOW) in gallons per minute are known, the NMGAS equation can be modified as

$$NMGAS = \frac{\% Gas - in - Mud * FLOW * 14.71}{B {S}^{2} * ROP} \qquad ( 11 )$$

Equivalent oil volume at surface (EOV) in ft³ can be computed from normalized gas volume as

$$E O V = \frac{NMGAS * 5.61}{G O R} \qquad ( 12 )$$

where 5.61 is the conversion factor between ft³ and barrels and GOR is gas-oil-ratio in ft3/bbl. Thus, bulk volume of hydrocarbons (BVHC) in an oil-bearing formation can be computed from equations (7) and (12) as

$$B V H C = \frac{E O V * {B}_{o}}{{V}_{c}} \qquad ( 13 )$$

where Bₒ is the oil formation volume factor.

In dry gas reservoirs, bulk volume of hydrocarbons can be computed as

BVHC=NMGAS*B_{g} (14)

where B_{g} is the gas formation volume factor.

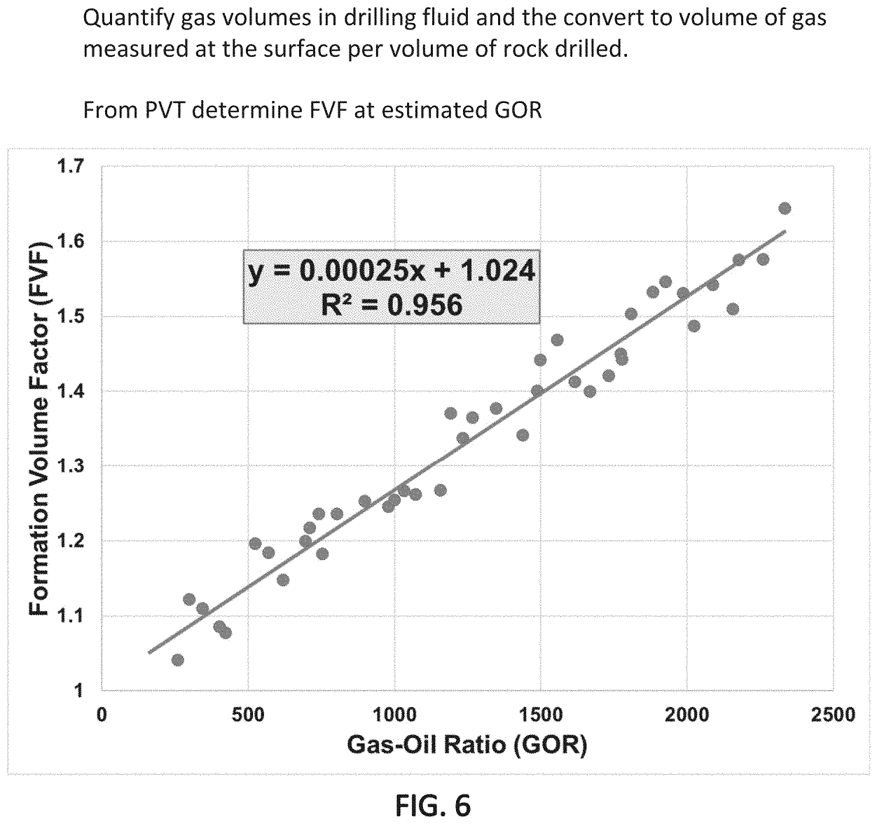

If the GOR is approximately known (from reservoir or regional fluid), formation volume factor can be estimated from GOR as shown in FIG. 6. Alternately, if GOR is unknown, it can be calculated from oil fraction as

Rel Wt. Oil=3070*[C₃C₅²]/[C₄\sqrt{C₂C₄}] (15)

Or if C₅ fraction isn't monitored, then oil fraction as

Rel Wt. Oil=1932*C₄²/\sqrt{C₂C₃} (16)

And estimated GOR(scf/stb) is =100,000*(Σᵢ₌₁⁴Cᵢ)/(Relative Wt. Oil) (17)

If pentane (C₅) is present, both equations 15 and 16 can be used to compute GOR. Usually, pentane concentration in the mud is small and extraction efficiency is low for unheated systems, which makes accurate measurement difficult. Heavier gases also tend to be recirculated to a greater extent, so background gas corrections are paramount.

Downhole PVT fluid samples can be used to calibrate and improve the characterization from mudgas. For example, Table 1 calculates RR by comparing gas trap values to reported fluid analysis from the same interval as the gas-in-air sample. analysis of downhole fluid samples. The mud gas value (ppm) is the gas value from the gas trap. The mud gas percent is the percent of each of the gases from the gas trap. The PVT results is the lab fluid value from the samples collected from a downhole fluid sampling tool. This tool collects C1 through C36. This fluid sample is from the same well at the same approximate depth as the Mud Gas Value from the gas trap. The PVT Percent-light gases only is the percent of that gas for C1 through C5 only. The Normalized Trap Response Factor (N-TRF) is calculated by dividing the Mud Gas Percent by the PVT Percent-light gases only. The Gas Trap Relative Response is finally then calculated by dividing each gas component by the N-TRF for methane.

TABLE 1
Shows the method in determining RR values from gas
values measured during drilling and comparing those
values to the light gases reported from PVT

| Gas Component | C1 | C2 | C3 | C4 | C5 |
|---|---|---|---|---|---|
| Mud gas Value PPM | 20366 | 744 | 235 | 39 | 15 |
| (Gas-in-Air) | | | | | |
| Mud gas percent from | 95.2% | 3.5% | 1.1% | 0.2% | 0.1% |
| gas trap | | | | | |
| PVT results (Mole %) | 57.2% | 5.7% | 3.8% | 2.3% | 1.4% |
| PVT Percent light | 81.3% | 8.1% | 5.4% | 3.2% | 2.0% |
| gases only | | | | | |
| N-TRF (mud | 1.17 | 0.43 | 0.20 | 0.06 | 0.03 |
| gas %/PVT %) | | | | | |
| Response (TRF Cx/ | 1.00 | 0.37 | 0.17 | 0.05 | 0.03 |
| TRF C1) | | | | | |

These gas trap relative response factors can also be used to correct the gas trap values measured in surrounding wells provided a similar drilling fluid was used. The method is helpful for estimating the relative response of the gas trap gas values relative to methane when no well site calibration runs are available.

At operation 18, pay cutoffs are determined. Cumulative pay summaries (HPV, net thickness, and flow capacity−perm*thickness) are generated from mud logs for each reservoir zone and compared with wireline logs with zonal multiplication factors for low-mid-high cases in field calibrated wells. These may be calculated, for example, by:

Calibrated net thickness from mud log=uncalibrated net thickness from mud log*(zonal multiplication factor)

Zonal multiplication factor=net thickness from wireline logs/uncalibrated net thickness from mud logs

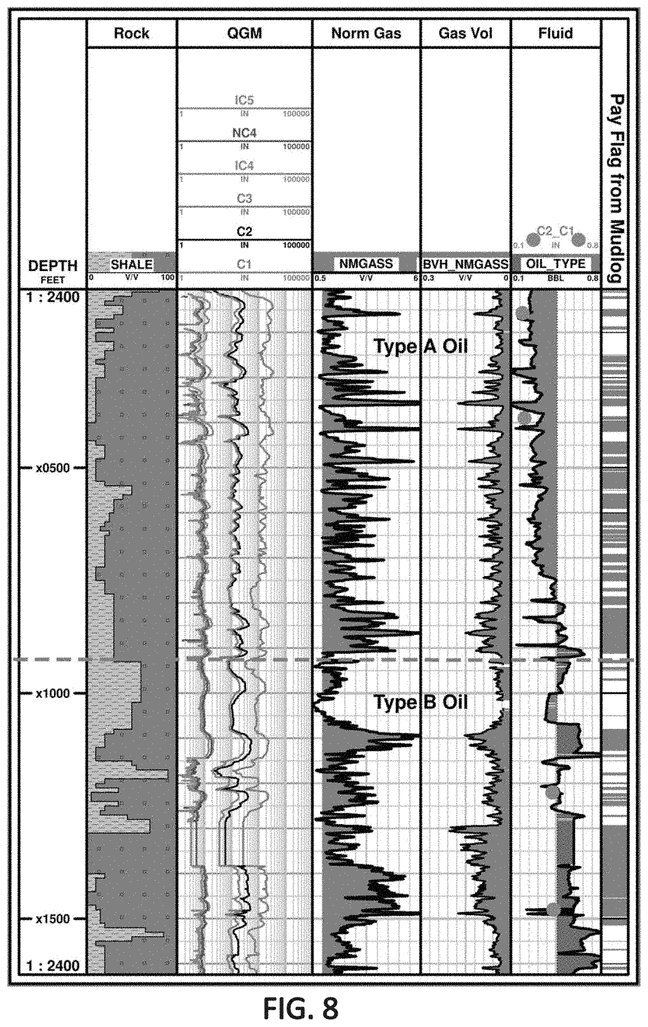

These values are used to calculate hydrocarbon in place, recoverable reserves, and productivity of wells. The technique can also be used to identify different oils depending on their C1/C2 ratios as shown in FIG. 8 which has a Type A oil and Type B oil. FIG. 8 illustrates the usefulness of the quantitative mud gas data as a tool for formation evaluation. The plot displays lithology, gas and bulk volume hydrocarbon (BVHC), and pay flags from the mudlog data. Additional analysis of PVT results identified two different oil types in the field. Both fluids have similar GOR and viscosities however there was a difference in methane values of the fluids. By plotting ratios of methane to ethane as illustrated in the “Fluid” track of the plot, the corrected mud gas data was used to identify the fluid types without the need to collect down hole fluid samples in development wells. Pay flag can be derived from mudlog as shown on track 6 (in this case, using a cutoff of normalized mud gas >1), and compared with pay flags from wireline/lwd interpretation.

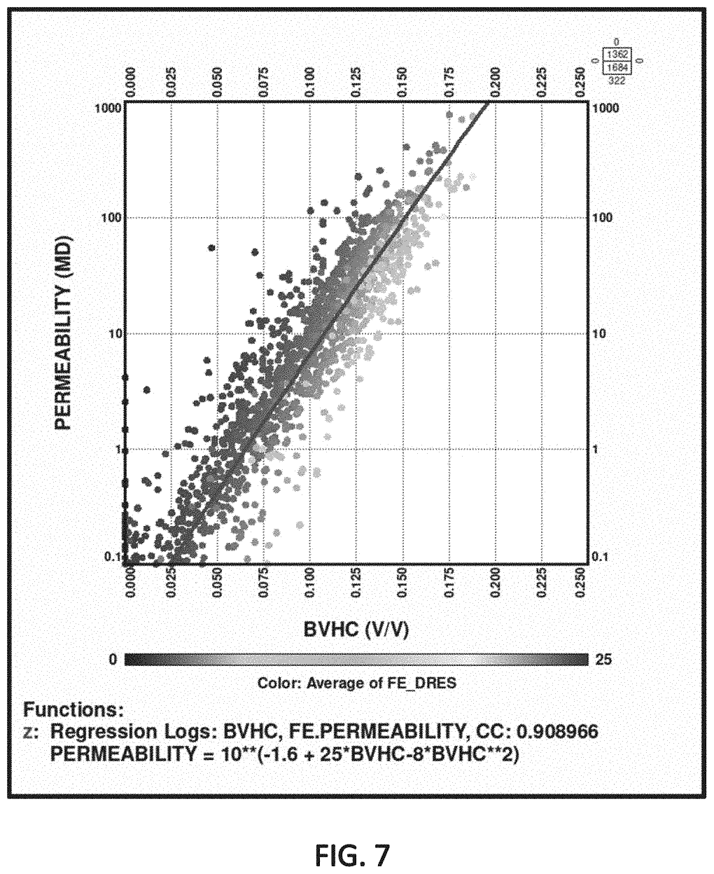

Operation 18 also estimates the permeability from the normalized gas volume. This may be done by computing a permeability vs bulk volume of hydrocarbons relation from wireline/LWD logs (calibrated to core data), as shown in FIG. 7. Thus, if bulk volume of hydrocarbons is calculated from mudlogs, the relation can be used to estimate a qualitative permeability from mudlogs. The permeability from mudlog doesn't compare well with the permeability from wireline logs on a depth-by-depth basis. However, permeability from mudlogs can be used to compute zonal averages which can be calibrated to wireline logs.

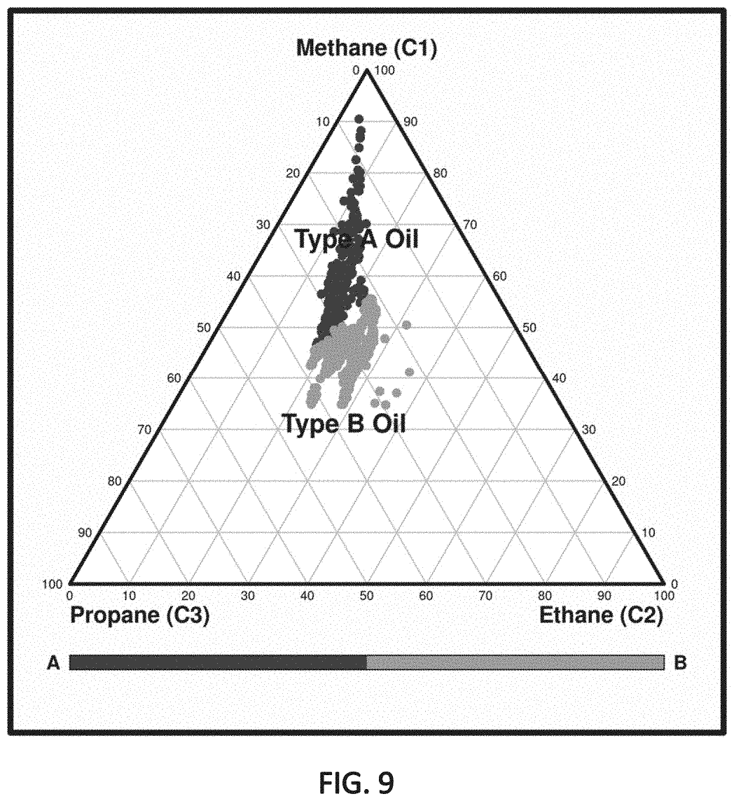

FIG. 9 shows a ternary plot approach of identifying different reservoir fluids from mudlogs. These ternary plots are commonly used in organic chemistry to identify minimum miscibility/critical point and we are adapting them for mudlog fluid typing. Instead of using a simple C2/C1 ratio, we can compare C1-C2-C3 and identify a gradation of fluid character changes. Lean gas reservoirs converge towards C1 and oil-bearing heavier reservoirs appear lower towards C2 and C3. In instances where C2/C1 ratio gives a false positive, the ternary plot can be used to confirm the fluid type. Additionally, the ternary can be easily modified to display C4 by plotting the ratio of C1/C4, C2/C4, and C3/C4.

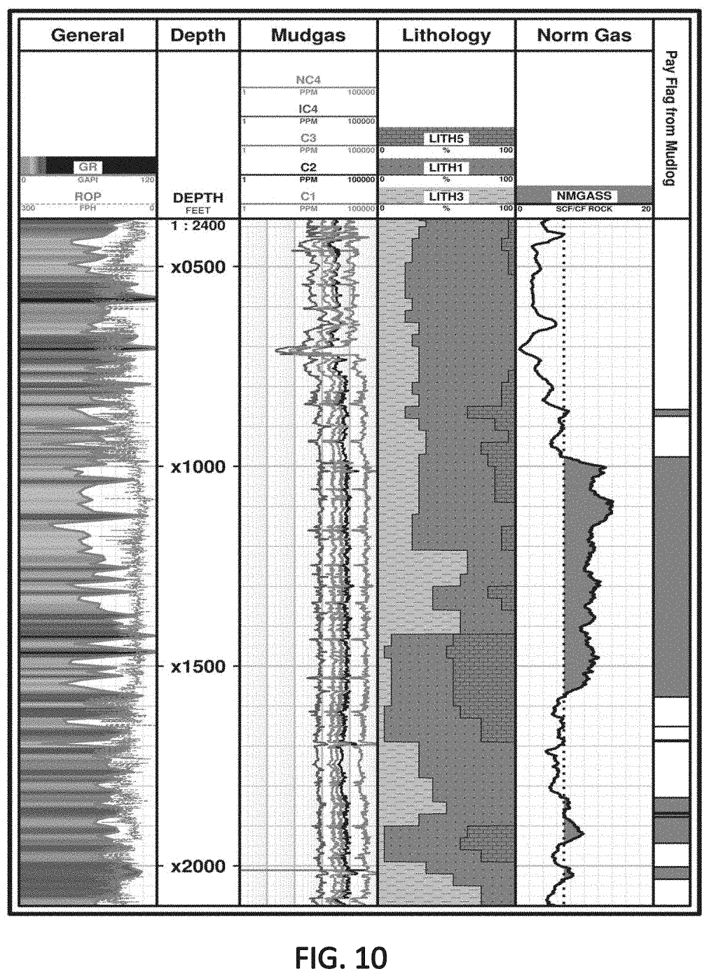

FIG. 10 displays mudlog data from an unconventional lateral well. Mudlogs are well suited for identifying high hydrocarbon intervals in development wells due to the very low permeability of these rocks. Typically, in high permeability reservoirs with overbalanced drilling conditions, a significant portion of the hydrocarbon can be pushed out of the rock ahead of the drill bit as it will not be circulated to the surface. By contrast, unconventional reservoirs have very low permeability and therefore, no significant volume of hydrocarbon is flushed ahead of the drill bit. Thus, NMGAS volumes can be used to estimate bulk volume of hydrocarbons if formation volume factor is known. Since downhole PVT samples are unlikely to be obtained in tight rocks, produced oil/gas at surface can be used to quantify formation volume factor. The track on the far right shows the pay flags resulting from method 100.

Method 100 may be tested by applying the technique in blind tests wells. If results within expected error range, the method can then be used in development wells without extensive wireline/LWD logs. This provides significant cost and time savings in the development wells.

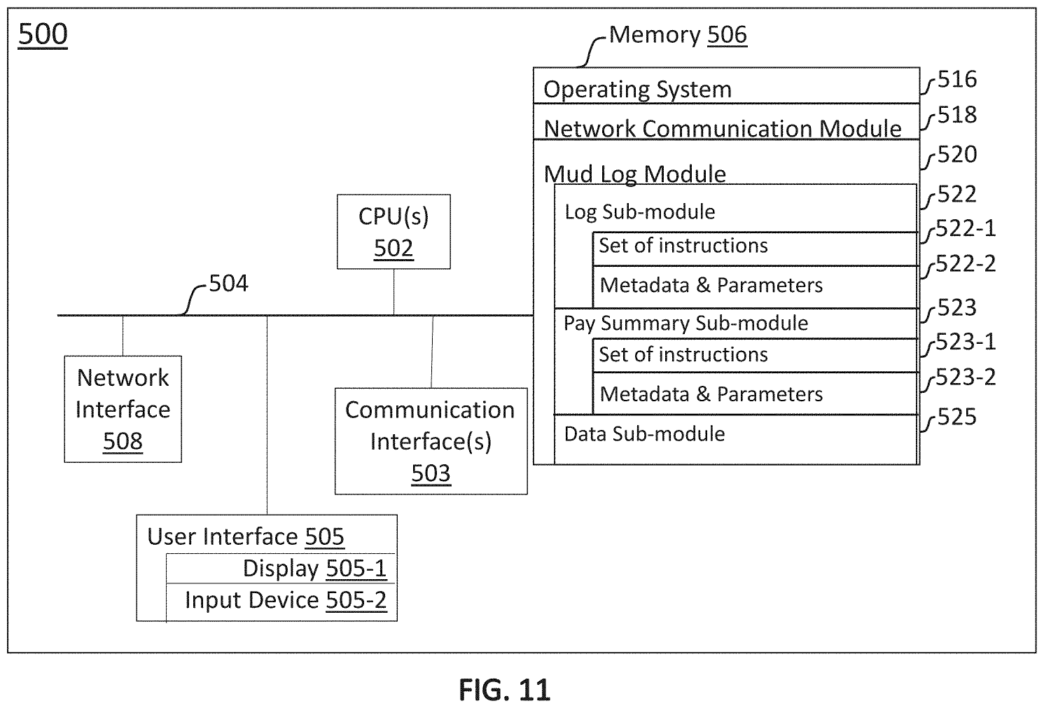

FIG. 11 is a block diagram illustrating a mud log pay determination system 500, in accordance with some embodiments. While certain specific features are illustrated, those skilled in the art will appreciate from the present disclosure that various other features have not been illustrated for the sake of brevity and so as not to obscure more pertinent aspects of the embodiments disclosed herein.

To that end, the mud log pay determination system 500 includes one or more processing units (CPUs) 502, one or more network interfaces 508 and/or other communications interfaces 503, memory 506, and one or more communication buses 504 for interconnecting these and various other components. The mud log pay determination system 500 also includes a user interface 505 (e.g., a display 505-1 and an input device 505-2). The communication buses 504 may include circuitry (sometimes called a chipset) that interconnects and controls communications between system components. Memory 506 includes high-speed random access memory, such as DRAM, SRAM, DDR RAM or other random access solid state memory devices; and may include non-volatile memory, such as one or more magnetic disk storage devices, optical disk storage devices, flash memory devices, or other non-volatile solid state storage devices. Memory 506 may optionally include one or more storage devices remotely located from the CPUs 502. Memory 506, including the non-volatile and volatile memory devices within memory 506, comprises a non-transitory computer readable storage medium and may store well logs, mud logs, and/or other information.

In some embodiments, memory 506 or the non-transitory computer readable storage medium of memory 506 stores the following programs, modules and data structures, or a subset thereof including an operating system 516, a network communication module 518, and a mud log module 520.

The operating system 516 includes procedures for handling various basic system services and for performing hardware dependent tasks.

The network communication module 518 facilitates communication with other devices via the communication network interfaces 508 (wired or wireless) and one or more communication networks, such as the Internet, other wide area networks, local area networks, metropolitan area networks, and so on.

In some embodiments, the mud log module 520 executes the operations of method 100. Mud log module 520 may include data sub-module 525, which handles the mud log and/or well log datasets. This data is supplied by data sub-module 525 to other sub-modules.

Log sub-module 522 contains a set of instructions 522-1 and accepts metadata and parameters 522-2 that will enable it to execute operations 10-17 of method 100. The pay summary sub-module 523 contains a set of instructions 523-1 and accepts metadata and parameters 523-2 that will enable it to execute to operation 18 of method 100. Although specific operations have been identified for the sub-modules discussed herein, this is not meant to be limiting. Each sub-module may be configured to execute operations identified as being a part of other sub-modules, and may contain other instructions, metadata, and parameters that allow it to execute other operations of use in processing data and generate the pay summary. For example, any of the sub-modules may optionally be able to generate a display that would be sent to and shown on the user interface display 505-1. In addition, any of the data or processed data products may be transmitted via the communication interface(s) 503 or the network interface 508 and may be stored in memory 506.

Method 100 is, optionally, governed by instructions that are stored in computer memory or a non-transitory computer readable storage medium (e.g., memory 506 in FIG. 11) and are executed by one or more processors (e.g., processors 502) of one or more computer systems. The computer readable storage medium may include a magnetic or optical disk storage device, solid state storage devices such as flash memory, or other non-volatile memory device or devices. The computer readable instructions stored on the computer readable storage medium may include one or more of: source code, assembly language code, object code, or another instruction format that is interpreted by one or more processors. In various embodiments, some operations in each method may be combined and/or the order of some operations may be changed from the order shown in the figures. For ease of explanation, method 100 is described as being performed by a computer system, although in some embodiments, various operations of method 100 are distributed across separate computer systems.

While particular embodiments are described above, it will be understood it is not intended to limit the invention to these particular embodiments. On the contrary, the invention includes alternatives, modifications and equivalents that are within the spirit and scope of the appended claims. Numerous specific details are set forth in order to provide a thorough understanding of the subject matter presented herein. But it will be apparent to one of ordinary skill in the art that the subject matter may be practiced without these specific details. In other instances, well-known methods, procedures, components, and circuits have not been described in detail so as not to unnecessarily obscure aspects of the embodiments.

The terminology used in the description of the invention herein is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, operations, elements, and/or components, but do not preclude the presence or addition of one or more other features, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “in response to determining” or “in accordance with a determination” or “upon detecting” or “in response to detecting” that the stated condition precedent is true, depending on the context.

Although some of the various drawings illustrate a number of logical stages in a particular order, stages that are not order dependent may be reordered and other stages may be combined or broken out. While some reordering or other groupings are specifically mentioned, others will be obvious to those of ordinary skill in the art and so do not present an exhaustive list of alternatives. Moreover, it should be recognized that the stages could be implemented in hardware, firmware, software or any combination thereof.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the invention and its practical applications, to thereby enable others skilled in the art to best utilize the invention and various embodiments with various modifications as are suited to the particular use contemplated.

What is claimed is:

1. A computer-implemented method of quantitative net pay determination and fluid determination from mud logs, comprising:
a. receiving, at one or more computer processors, at least one mud log from at least one well drilled through a subterranean rock formation containing hydrocarbons;
b. selecting, via a graphical user interface, extraction system type, operating, and drilling parameters for the at least one mud log;
c. determining, via the one or more computer processors, hydrocarbon parameters including at least one of methane (C1) extraction efficiency, trap response factor (TRF), and relative responses for ethane-pentane (C2-C5);
d. correcting, via the one or more computer processors, gas-in-mud volume to an earth surface for the at least one mud log to generate a corrected C1-C5;
e. calculating, via the one or more computer processors, a gas-to-oil ratio (GOR) from the corrected C1-C5;
f. calculating, via the one or more computer processors, a reservoir gas volume from the gas-in-mud volume to an earth surface and the GOR;
g. determining pay cutoffs based on the reservoir gas volume; and
h. generating, via the one or more computer processors, at least one pay summary for the at least one well based on the pay cutoffs.

2. The method of claim 1 wherein the determining the hydrocarbon parameters is done using a calibration run.

3. The method of claim 1 wherein the determining the hydrocarbon parameters is done using empirical values.

4. The method of claim 1 wherein the calculating the gas reservoir volume is done using pressure-volume-temperature data for field fluids.

5. The method of claim 1 wherein the calculating the gas reservoir volume is done using regional fluid data.

6. The method of claim 1 further comprising estimating permeability from the reservoir gas volume.

7. The method of claim 1 further comprising generating three pay summaries for a low case, mid case, and high case.

8. The method of claim 1 further comprising real-time fluid typing.

9. A computer system, comprising:
one or more processors;
memory; and
one or more programs, wherein the one or more programs are stored in the memory and configured to be executed by the one or more processors, the one or more programs including instructions that when executed by the one or more processors cause the system to:
a. receive, at the one or more processors, at least one mud log from at least one well drilled through a subterranean rock formation containing hydrocarbons;
b. select, via a graphical user interface, extraction system type, operating, and drilling parameters for the at least one mud log;
c. determine, via the one or more processors, hydrocarbon parameters including at least one of methane (C1) extraction efficiency, trap response factor (TRF), and relative responses for ethane-pentane (C2-C5);
d. correct, via the one or more processors, gas-in-mud volume to an earth surface for the at least one mud log to generate a corrected C1-C5;
e. calculate, via the one or more processors, a gas-to-oil ratio (GOR) from the corrected C1-C5;
f. calculate, via the one or more processors, a reservoir gas volume from the gas-in-mud volume to an earth surface and the GOR;
g. determining pay cutoffs based on the reservoir gas volume; and
h. generate, via the one or more computer processors, at least one pay summary for the at least one well based on the pay cutoffs.

10. The computer system of claim 9 further comprising displaying the at least on pay summary on the graphical user interface.

11. A non-transitory computer readable storage medium storing one or more programs, the one or more programs comprising instructions, which when executed by an electronic device with one or more processors and memory, cause the device to:
a. receive, at the one or more processors, at least one mud log from at least one well drilled through a subterranean rock formation containing hydrocarbons;
b. select, via a graphical user interface, extraction system type, operating, and drilling parameters for the at least one mud log;
c. determine, via the one or more processors, hydrocarbon parameters including at least one of methane (C1) extraction efficiency, trap response factor (TRF), and relative responses for ethane-pentane (C2-C5);
d. correct, via the one or more processors, gas-in-mud volume to an earth surface for the at least one mud log to generate a corrected C1-C5;
e. calculate, via the one or more processors, a gas-to-oil ratio (GOR) from the corrected C1-C5;
f. calculate, via the one or more processors, a reservoir gas volume from the gas-in-mud volume to an earth surface and the GOR;
g. determining pay cutoffs based on the reservoir gas volume; and
h. generate, via the one or more computer processors, at least one pay summary for the at least one well based on the pay cutoffs.

12. The computer system of claim 9 further comprising displaying the at least on pay summary on the graphical user interface.
